(19) United States
(12) Patent Application Publication
(10) Pub. No.: US 2013/0125914 A1
(43) Pub. Date: May 23, 2013

(54) HAIR-CONDITIONING COMPOSITIONS

(71) Applicant: Henkel AG & Co. KGaA, Duesseldorf (DE)

(72) Inventors: Marlene Battermann, Asendorf (DE); Petra Westphal, Neu Wulmstorf (DE)

(73) Assignee: Henkel AG & Co. KGaA, Duesseldorf (DE)

(21) Appl. No.: 13/740,401

(22) Filed: Jan. 14, 2013

Related U.S. Application Data
(63) Continuation of application No. PCT/EP2011/058446, filed on May 24, 2011.

(30) Foreign Application Priority Data
Jul. 14, 2010 (DE) 10 2010 031 318.1

(51) Int. Cl. A61K 8/891; A61Q 5/12; A61K 8/49

(52) U.S. Cl. CPC A61K 8/891; A61K 8/4946; A61Q 5/12
USPC 132/202; 424/70.12

(57) ABSTRACT

Hair treatment agents contain selected alcohols and selected silicones, and are used for treating hair. The agents are preferably packaged as a non-aerosol spray application.

### FIELD OF THE INVENTION

The present invention generally relates to hair treatment agents containing selected alcohols and selected silicones, and to the use of said agents for treating hair Said agents are preferably packaged as a non-aerosol spray application.

### BACKGROUND OF THE INVENTION

Not least because of the heavy loading on the hair, resulting for example from coloring or permanent waving and from washing the hair with shampoos as well as from environmental pollution, the importance of care products having as long-lasting an effect as possible is growing.

These conventional compositions contain cyclomethicones. As a consequence of this, some active ingredients cannot penetrate into the interior of the hair or cannot do so deeply enough. The use of the products, which are frequently offered as a spray version, can cause irritation of the airways in the user. These irritations can be provoked by cyclomethicones and short-chain alcohols. In hair styling, moreover, a composition having a high silicone content makes hair styling more troublesome and difficult. A full hairstyle with easy combability of the wet and dry hair is not obtained with compositions of the prior art.

There is therefore still a need for active ingredients or active ingredient combinations for cosmetic agents with good care properties and good biodegradability, which are both free from cyclomethicones and free from short-chain alcohols.

An object of the present invention is therefore that in order to achieve the desired effect the composition should be distributed quickly and uniformly on the treated hair. A further object consists in improving the tolerability of the compositions. A further object consists in further increasing the conditioning effects of the conventional compositions.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic composition for treating human hair, containing at least 0.01 wt. % of a cationic imidazoline derivative of the 4,5-dihydro-1-(C1 to C4 alkyl)-2-(C12 to C30 alkyl)-1-(2-(C12-C30 alkyl) amidoethyl) imidazolinium salt type, in particular those having a methyl, ethyl, propyl or butyl group as the C1 to C4 alkyl, and having in each case as the C12 to C30 alkyl residue, independently of one another but most highly preferably simultaneously, an alkyl residue of at least 20 C atoms, and particularly preferably of at least 21 C atoms; at least one silicone selected from the dimethicones, dimethiconols and amodimethicones; and a cosmetic carrier containing in addition to water an alcohol selected from glycol, glycerol, 1,2-pentanediol, 1,5-pentanediol, 1,2-hexanediol, 1,6-hexanediol, benzyl alcohol, phenoxyethanol or mixtures thereof.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly it has been found that cationic imidazolines of the 4,5-dihydro-1-(C1 to C4 alkyl)-2-(C12 to C30 alkyl)-1-(2-(C12-C30 alkyl) amidoethyl) imidazolinium methosulfate type, in particular those having a methyl, ethyl, propyl or butyl group as the C1 to C4 alkyl group and having in each case as the C12 to C30 alkyl residue, independently of one another but most highly preferably simultaneously, an alkyl residue of at least 20 C atoms, and particularly preferably 21 C atoms, when [combined] with silicones selected from the dimethicones, dimethiconols and/or amodimethicones in a cosmetic carrier containing in addition to water an alcohol selected from glycol, glycerol, 1,2-pentanediol, 1,5-pentanediol, 1,2-hexanediol, 1,6-hexanediol, benzyl alcohol, phenoxyethanol or mixtures thereof, achieve the object in an outstanding manner.

The present invention therefore firstly provides a composition for treating human hair, containing
a) at least 0.01 wt. % of a cationic imidazoline derivative of the 4,5-dihydro-1-(C1 to C4 alkyl)-2-(C12 to C30 alkyl)-1-(2-(C12-C30 alkyl) amidoethyl) imidazolinium salt type, in particular those having a methyl, ethyl, propyl or butyl group as the C1 to C4 alkyl, and having in each case as the C12 to C30 alkyl residue, independently of one another but most highly preferably simultaneously, an alkyl residue of at least 20 C atoms, and particularly preferably 21 C atoms,
b) at least one silicone selected from the dimethicones, dimethiconols and amodimethicones and
c) a cosmetic carrier containing in addition to water an alcohol selected from glycol, glycerol, 1,2-pentanediol, 1,5-pentanediol, 1,2-hexanediol, 1,6-hexanediol, benzyl alcohol, phenoxyethanol or mixtures thereof.

Hair treatment agents within the meaning of the present invention are for example hair coloring agents, bleaching agents, hair shampoos, hair conditioners, conditioning shampoos, hair sprays, hair rinses, hair masks, hair packs, hair tonics, permanent wave fixing solutions, hair coloring shampoos, hair coloring agents, hair fixing agents, hair setting agents, hair styling preparations, blow-drying lotions, styling mousses, hair gels, hair waxes or combinations thereof.

According to the invention combability is understood to mean both the combability of wet fibers and the combability of dry fibers. The combing work applied or the force applied during combing of a group of fibers serves as a measure of combability. The measurement parameters can be assessed by sensory means by the person skilled in the art or quantified by means of measuring devices.

Feel is defined as the tactility of a group of fibers, the person skilled in the art feeling and assessing the parameters fullness and softness of the group of fibers by sensory means.

Shaping is understood to be the ability to change the shape of a group of previously treated keratin-containing fibers, in particular human hair. In hair cosmetics this is also referred to as styleability.

According to the invention an oxidative hair treatment is defined as the action of an oxidative cosmetic agent containing at least one oxidizing agent in a cosmetic carrier on hair.

The ingredients are described in detail below. Where reference is made below to the active ingredient complex (A), this relates to the ingredients mandatorily contained in the agents according to the invention.

O/W, W/O and W/O/W emulsions in the form of creams or gels or surfactant-containing foaming solutions, such as for example shampoos, foam aerosols or other preparations that are suitable in particular for use on the hair, are particularly suitable according to the invention as cosmetic carriers c). The cosmetic carrier c) contains at least 50 wt. % of water. Furthermore, within the meaning of the present invention the cosmetic carrier c) contains an alcohol selected from glycol, glycerol, 1,2-pentanediol, 1,5-pentanediol, 1,2-hexanediol, 1,6-hexanediol, benzyl alcohol, phenoxyethanol or mixtures thereof. The agents according to the invention can additionally contain further organic solvents, such as for example methoxybutanol, ethyl diglycol or 1,2-propylene glycol. All water-soluble organic solvents are preferred here.

Carriers containing 0.1 to 20 wt. % of the alcohol selected from glycol, glycerol, 1,2-pentanediol, 1,5-pentanediol, 1,2-hexanediol, 1,6-hexanediol, benzyl alcohol, phenoxyethanol or mixtures thereof are preferred. The alcohols selected from glycol, glycerol, benzyl alcohol and/or phenoxyethanol and mixtures thereof are preferred. Particularly preferred carriers contain the selected alcohols in the following quantity ranges: glycol from 0.1 to 5 wt. %, phenoxyethanol from 0.1 to 5.0 wt. %, preferably 0.3 to 3.0 wt. %, glycerol from 0.1 to 10 wt. %, preferably 0.5 to 5.0 wt. % and benzyl alcohol from 0.1 to 10 wt. %. Most highly preferred carriers contain both phenoxyethanol and glycerol in the aforementioned quantity ranges. Compositions according to the invention containing in addition to the explicitly mentioned alcohols no alcohols such as methanol, ethanol, isopropanol, propanol or butanols are most highly preferred.

As ingredient a) the agents according to the invention contain at least one quaternary imidazoline compound, i.e. a compound having a positively charged imidazoline ring. The imidazoline compounds according to the invention are cationic imidazolines of the 4,5-dihydro-1-(C1 to C4 alkyl)-2-(C12 to C30 alkyl)-1-(2-(C12-C30 alkyl) amidoethyl) imidazolinium salt type, in particular those having a methyl, ethyl, propyl or butyl group as the C1 to C4 alkyl group, and having in each case as the C12 to C30 alkyl residue, independently of one another but most highly preferably simultaneously, an alkyl residue of at least 20 C atoms, and particularly preferably 21 C atoms. Formula I shows the structure of these compounds.

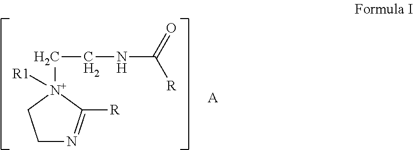

The residues R independently of one another denote a saturated or unsaturated, linear or branched hydrocarbon residue having a chain length of 12 to 30 carbon atoms. The preferred compounds of formula I each contain the same hydrocarbon residue for R. The chain length of the residues R is preferably at least 20 carbon atoms. Compounds having a chain length of at least 21 carbon atoms are particularly preferred. The residue R1 denotes a methyl, ethyl, propyl, isopropyl, butyl, isobutyl or tert-butyl group. R1 is preferably methyl or ethyl. R1 is particularly preferably a methyl group. A denotes a physiologically tolerable anion. Halides, for example fluoride, chloride or bromide, alkyl sulfates, such as, methosulfate or ethosulfate, phosphates, citrate, tartrate, maleate or fumarate are encompassed according to the invention as the anionic counterion. A commercial product of this chain length is known for example under the name Quatemium-91.

The imidazolines of formula I are contained in the compositions according to the invention in amounts from 0.01 to 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the overall composition of the individual agent.

The silicone as ingredient b) according to claim 1 is selected from the group of dimethicones and/or the group of dimethiconols and/or the group of amino-functional silicones.

According to the invention the compositions according to the invention particularly preferably contain no cyclomethicones in addition to the selected silicones.

The dimethicones according to the invention can be both linear and branched. Linear dimethicones can be represented by the following structural formula (Si1):

(SiR¹₃)—O—(SiR²₂—O—)ₓ—(SiR¹₃) (Si1)

Branched dimethicones can be represented by the structural formula (Si1.1):

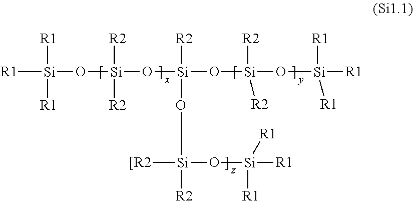

Residues R1 and R2 independently of one another denote hydrogen, a methyl residue, a C2 to C30 linear, saturated or unsaturated hydrocarbon residue, a phenyl residue and/or an aryl residue. The numbers x, y and z are whole numbers and each run independently of one another from 0 to 50,000. The molecular weights of the dimethicones are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs. Viscosities around the range of approximately 60,000 cPs are most highly preferred. Reference is made here by way of example to the product “Dow Corning 200 with 60,000 cSt”. It is most preferable according to the invention for the dimethicones according to the invention already to be in the form of emulsion-polymerized dimethicones. Emulsion-polymerized dimethicones can be used either as a mechanical pre-emulsion of a dimethicone or as an emulsion of a dimethicone prepared by the emulsion polymerization method. The latter is most preferred. An example of such most highly preferred emulsions of dimethicones obtained by the emulsion polymerization method is the commercial product Dow Corning® 1664, which is also known under the name Xiameter® MEM-1664.

Further dimethicones according to the invention have the characterizing feature that they contain at least one silicone of formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are contained in the compositions according to the invention in amounts from 0.01 to 10 wt. %, preferably 0.01 to 8 wt. %, particularly preferably 0.1 to 7.5 wt. % and in particular 0.1 to 5 wt. %, relative to the overall composition.

Dimethiconols according to the invention can be both linear and branched. Linear dimethiconols can be represented by the following structural formula (Si8-I):

(SiOHR¹₂)—O—(SiR²₂—O—)ₓ—(SiOHR¹₂) (Si8-1)

Branched dimethiconols can be represented by the structural formula (Si8-II):

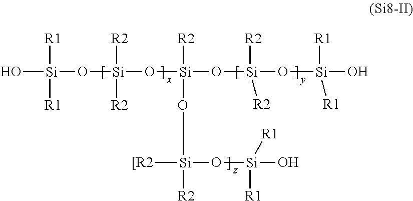

Residues R¹ and R² independently of one another denote hydrogen, a methyl residue, a C2 to C30 linear, saturated or unsaturated hydrocarbon residue, a phenyl residue and/or an aryl residue. The numbers x, y and z are whole numbers and each run independently of one another from 0 to 50,000. The molecular weights of the dimethiconols are between 1000 D and 10,000,000 D. The viscosities are between 100 and 10,000,000 cPs, measured at 25° C. with a glass capillary viscometer in accordance with the Dow Corning corporate test method CTM 0004 of 20 Jul. 1970. Preferred viscosities are between 1000 and 5,000,000 cPs, most particularly preferred viscosities are between 10,000 and 3,000,000 cPs. The most preferred range is between 50,000 and 2,000,000 cPs.

The following commercial products are cited as examples of such products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (these last four all GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (these last all Wacker-Chemie GmbH).

The use of emulsion-polymerized dimethiconols is most highly preferred. Emulsion-polymerized dimethiconols can be used either as a mechanical pre-emulsion of a dimethiconol or as an emulsion of a dimethiconol prepared by the emulsion polymerization method. The latter is most preferred. Examples of such most highly preferred emulsions of dimethiconols obtained by the emulsion polymerization method are the commercial products X2-1766 and X2-1784 from Dow Corning.

The dimethiconols (Si8) are contained in the compositions according to the invention in amounts from 0.01 to 10 wt. %, preferably 0.01 to 8 wt. %, particularly preferably 0.1 to 7.5 wt. % and in particular 0.1 to 5 wt. % of dimethiconol, relative to the composition.

Amino-functional silicones according to the invention can be described for example by formula (Si-2)

M(RₐQ_{b}SiO_{(4-a-b)/2})ₓ(R_{c}SiO_{(4-c)/2})_{y}M (Si-2)

in which in the above formula
R is a hydrocarbon or a hydrocarbon residue having 1 to approximately 6 carbon atoms,
Q is a polar residue of the general formula —R¹HZ,
   in which
   R¹ is a divalent linking group that is bonded to hydrogen and to the residue Z, composed of carbon and hydrogen atoms, carbon, hydrogen and oxygen atoms or carbon, hydrogen and nitrogen atoms, and
   Z is an organic, amino-functional residue containing at least one amino-functional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2000, preferably from approximately 3 to approximately 50 and most preferably from approximately 3 to approximately 25, and
y is a number in the range from approximately 20 to approximately 10,000, preferably from approximately 125 to approximately 10,000 and most preferably from approximately 150 to approximately 1000, and
M is a suitable silicone end group as is known in the prior art, preferably trimethylsiloxy.

According to formula (Si-2) Z is an organic, amino-functional residue containing at least one functional amino group. A possible formula for said Z is NH(CH₂)_{z}NH₂, in which z is a whole number greater than or equal to 1. Another possible formula for said Z is —NH(CH₂)_{z}(CH₂)_{zz}NH, in which both z and zz are independently of one another a whole number greater than or equal to 1, this structure encompassing diamino ring structures, such as piperazinyl. Said Z is most preferably an —NHCH₂CH₂NH₂ residue. Another possible formula for said Z is —N(CH₂)_{z}(CH₂)_{zz}NX₂ or —NX₂, in which each X of X₂ is selected independently from the group consisting of hydrogen and alkyl groups having 1 to 12 carbon atoms, and zz is 0.

Q according to formula (Si-2) is most preferably a polar amino-functional residue of the formula —CH₂CH₂CH₂NHCH₂CH₂NH₂.

In formula (Si-2) a assumes values in the range from 0 to 2, b assumes values in the range from 2 to 3, a+b is less than or equal to 3, and c is a number in the range from 1 to 3. Cationic silicone oils such as for example the commercially available products Dow Corning (DC) 929 Emulsion, DC2-2078, DC5-7113, SM-2059 (General Electric) and SLM-55067 (Wacker) are suitable according to the invention.

Particularly preferred agents according to the invention have the characterizing feature that they contain at least one amino-functional silicone of formula (Si-3a)

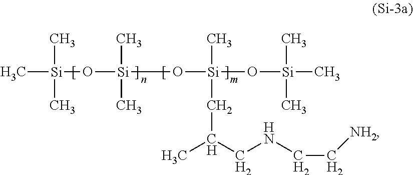

in which m and n are numbers whose sum (m+n) is between 1 and 2000, preferably between 50 and 150, wherein n preferably assumes values from 0 to 1999 and in particular from 49 to 149 and m preferably assumes values from 1 to 2000, in particular from 1 to 10.

Under the INCI declaration these silicones are known as trimethylsilylamodimethicones and they are available for example under the name Q2-7224 (manufacturer: Dow Corning; a stabilized trimethylsilylamodimethicone).

Agents according to the invention are also particularly preferred that contain an amino-functional silicone of formula (Si-3b)

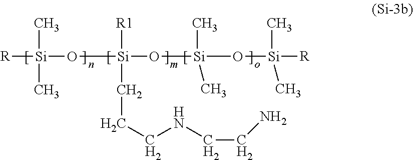

in which
R denotes —OH, an (optionally ethoxylated and/or propoxylated) (C₁ to C₂₀) alkoxy group or a —CH₃ group,
R1 denotes —OH, a (C₁ to C₂₀) alkoxy group or a —CH₃ group, and
m, n and o are numbers whose sum (m+n+o) is between 1 and 2000, preferably between 50 and 150, wherein the sum (n+o) preferably assumes values from 0 to 1999 and in particular from 49 to 149 and m preferably assumes values from 1 to 2000, in particular from 1 to 10.

Under the INCI declaration these silicones are known as amodimethicones or as functionalized amodimethicones, such as for example Bis(C13-15 Alkoxy) PG Amodimethicone (available for example as the commercial product DC 8500 from Dow Corning), Trideceth-9 PG-Amodimethicone (available for example as the commercial product Silcare® Silicone SEA from Clariant). Suitable diquaternary silicones are selected from compounds of the general formula (Si3c)

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c)

in which residues R1 to R6 independently of one another denote C1 to C22 alkyl residues, which can contain hydroxyl groups, and wherein preferably at least one of the residues has at least 8 C atoms and the other residues have 1 to 4 C atoms,
residues R7 to R12 independently of one another are identical or different and denote C1 to C10 alkyl or phenyl, A denotes a divalent organic group of compounds,
n is a number from 0 to 200, preferably from 10 to 120, particularly preferably from 10 to 40,
and X⁻ is an anion.

The divalent group of compounds is preferably a C1 to C12 alkylene or alkoxyalkylene group, which can be substituted with one or more hydroxyl groups. The group —(CH2)3-O—CH2-CH(OH)—CH2- is particularly preferred.

The anion X— can be a halide ion, an acetate, an organic carboxylate or a compound of the general formula RSO3-, in which R has the meaning of C1 to C4 alkyl residues.

A further group of amino-functional silicones is known under the INCI name Silicone Quaternium-xx, wherein xx denotes a number, for example 2, 3, 4, 5, 6, 7, 8, 10, 12, 14, 18, 20, 22 etc. A group of these silicone compounds is derived from dimethicone copolyols or dimethiconol copolyols. According to the invention dimethicone copolyols are understood to be preferably polyoxyalkylene-modified dimethyl polysiloxanes of the general formulae (Si-6) or (Si-7):

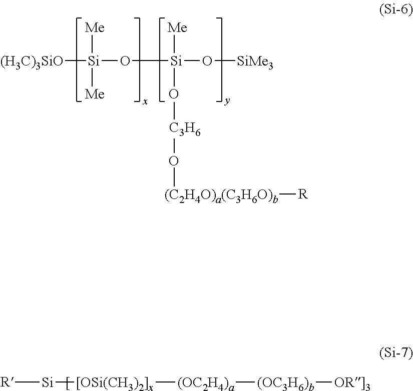

In the case of Silicone Quaternium-xx types the residues R in these structures contain quaternary nitrogens, amino-functional residues and/or amidoamines as quaternary residues. The exact structures can be obtained from the manufacturers' data sheets. One example of such a product is the commercial product SilSense® Q Plus from Lubrizol. Further examples are available commercially from Dow Corning under the trade name Dow Corning 5-7113 (Silicone Quaternium-16), Dow Corning 5-7070 (Silicone Quatemium-16/Glycidoxy Dimethicone Crosspolymer) and also Silquat Polyether Fatty Quats type Siltech AD (Silicone Quaternium-8), Silquat AC and Silquat D208, as well as silicones of the SilCare® Silicone SEA type.

The following structural formula represents further examples of silicone quaternium types.

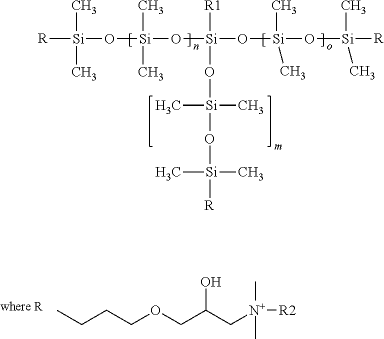

The anion can be a halide ion, an acetate, an organic carboxylate or a compound of the general formula RSO₃⁻, in which R has the meaning of C1 to C4 alkyl residues.

Such amino-functional silicones are sold under the trade name Abilquat T-60 or the INCI name Silicone Quatemium-22.

A preferred diquaternary silicone has the general formula (Si3d)

[RN⁺Me₂-A-(SiMe₂O)ₙ—SiMe₂-A-N⁺Me₂R]2CH₃COO (Si3d),

in which A is the group —(CH₂)₃—O—CH₂—CH(OH)—CH₂—,
R is an alkyl residue having at least 8 C atoms and n is a number from 10 to 120.

Suitable silicone polymers having two terminal, quaternary ammonium groups are known under the INCI name Quaternium-80. These are dimethyl siloxanes having two terminal trialkylammonium groups. Such diquaternary polydimethyl siloxanes are sold by Evonik under the trade names Abil® Quat 3270, 3272 and 3474.

Preparations that are preferred according to the invention have the characterizing feature that they contain, relative to their weight, 0.01 to 10 wt. %, preferably 0.01 to 8 wt. %, particularly preferably 0.1 to 7.5 wt. % and in particular 0.2 to 5 wt. % of amino-functional silicone(s) and/or diquaternary silicone.

The compositions according to the invention can contain at least one polyammonium-polysiloxane compound as the silicone. The polyammonium-polysiloxane compounds can be purchased for example from GE Bayer Silicones under the trade name Baysilone®. The products with the names Baysilone TP 3911, SME 253 and SFE 839 are preferred here. The use of Baysilone TP 3911 as the active component of the compositions according to the invention is most particularly preferred. The polyammonium-polysiloxane compounds are used in the compositions according to the invention in an amount from 0.01 to 10 wt. %, preferably 0.01 to 7.5, particularly preferably 0.01 to 5.0 wt. %, most particularly preferably from 0.05 to 2.5 wt. %, relative in each case to the overall composition.

Ester oils are used as a further particularly advantageous ingredient for the present invention. Ester oils are defined as follows:

Ester oils are understood to be the esters of C₆-C₃₀ fatty acids with C₂-C₃₀ fatty alcohols. The monoesters of fatty acids with alcohols having 2 to 24 C atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof.

Particularly preferred according to the invention are isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

The ester oils can naturally also be alkoxylated with ethylene oxide, propylene oxide or mixtures of ethylene oxide and propylene oxide. The alkoxylation can take place both on the fatty alcohol part and on the fatty acid part and also on both parts of the ester oils. It is preferred according to the invention, however, for the fatty alcohol to be alkoxylated first and then esterified with fatty acid. These compounds are shown in general in formula (D4-II).

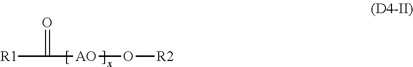

R1 here denotes a saturated or unsaturated, branched or unbranched, cyclic saturated, cyclic unsaturated acyl residue having 6 to 30 carbon atoms,
AO denotes ethylene oxide, propylene oxide or butylene oxide,
X denotes a number between 1 and 200, preferably between 1 and 100, particularly preferably between 1 and 50, most particularly preferably between 1 and 20, most highly preferably between 1 and 10 and most preferably between 1 and 5,
R2 denotes a saturated or unsaturated, branched or unbranched, cyclic saturated, cyclic unsaturated alkyl, alkenyl, alkynyl, phenyl or benzyl residue having 6 to 30 carbon atoms. Examples of fatty acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components as residue R2 in the ester oils are benzyl alcohol, isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. An ester oil that is particularly preferred according to the invention is available for example under the INCI name PPG-3 Benzyl Ether Myristate, for example the commercial product Crodamol® STS.

Ester oils are also understood to include:
Dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate, and
Symmetrical, asymmetrical or cyclic esters of carbonic acid with fatty alcohols, for example glycerol carbonate or dicaprylyl carbonate (Cetiol® CC),
Tri-fatty acid esters of saturated and/or unsaturated linear and/or branched fatty acids with glycerol,
Fatty acid partial glycerides, namely monoglycerides, diglycerides and technical mixtures thereof. If technical products are used, small amounts of triglycerides may also be included for production reasons. The partial glycerides preferably obey formula (D4-I),

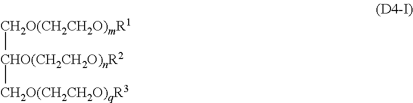

in which R1, R2 and R3 independently of one another denote hydrogen or a linear or branched, saturated and/or unsaturated acyl residue having 6 to 22, preferably 12 to 18, carbon atoms, with the proviso that at least one of these groups denotes an acyl residue and at least one of these groups denotes hydrogen. The sum (m+n+q) denotes 0 or numbers from 1 to 100, preferably 0 or 5 to 25. R1 preferably denotes an acyl residue and R2 and R3 hydrogen and the sum (m+n+q) is preferably 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Oleic acid monoglycerides are preferably used.

The ester oils are used in the agents according to the invention in an amount from 0.01 to 20 wt. %, preferably 0.01 to 10.0 wt. %, particularly preferably 0.01 to 7.5 wt. %, most highly preferably from 0.1 to 5.0 wt. %. Naturally it is also possible according to the invention to use more than two ester oils at the same time. It has proved according to the invention to be particularly advantageous for a fast-spreading ester oil to be combined with a slow-spreading ester oil.

At least one cationically charged polymeric compound is included as a further particularly advantageous ingredient. Cationic polymers are understood to be polymers having a group in the main and/or side chain which can be “temporarily” or “permanently” cationic.

Such polymers that have an adequate solubility in the aqueous carrier according to the invention so as to completely dissolve in the agent according to the invention are preferred.

The cationic polymers can be homo- or copolymers, wherein the quaternary nitrogen groups are contained either in the polymer chain or preferably as a substituent at one or more of the monomers. The ammonium group-containing monomers can be copolymerized with non-cationic monomers. Suitable cationic monomers are unsaturated, radically polymerizable compounds bearing at least one cationic group, in particular ammonium-substituted vinyl monomers such as for example trialkyl methacryloxy alkylammonium, trialkyl acryloxy alkylammonium, dialkyl diallyl ammonium and quaternary vinyl ammonium monomers with cyclic groups containing cationic nitrogens, such as pyridinium, imidazolium or quaternary pyrrolidones, e.g. alkyl vinyl imidazolium, alkyl vinyl pyridinium, or alkyl vinyl pyrrolidone salts. The alkyl groups of these monomers are preferably low alkyl groups such as for example C1 to C7 alkyl groups, particularly preferably C1 to C3 alkyl groups.

The ammonium group-containing monomers can be copolymerized with non-cationic monomers. Suitable comonomers are for example acrylamide, methacrylamide, alkyl and dialkyl acrylamide, alkyl and dialkyl methacrylamide, alkyl acrylate, alkyl methacrylate, vinyl caprolactone, vinyl caprolactam, vinyl pyrrolidone, vinyl esters, for example vinyl acetate, vinyl alcohol, propylene glycol or ethylene glycol, the alkyl groups of these monomers preferably being C1 to C7 alkyl groups, particularly preferably C1 to C3 alkyl groups.

Suitable polymers having quaternary amine groups are for example the polymers described in the CTFA Cosmetic Ingredient Dictionary under the name Polyquaternium, such as methylvinyl imidazolium chloride/vinyl pyrrolidone copolymer (Polyquaternium-16) or quaternized vinyl pyrrolidone/dimethyl aminoethyl methacrylate copolymer (Polyquaternium-11).

Of the cationic polymers that can be included in the agent according to the invention, vinyl pyrrolidone/dimethylaminoethyl methacrylate methosulfate copolymer for example, which is sold under the trade names Gafquat® 755 N and Gafquat® 734 by Gaf Co., USA, is suitable, of which Gafquat® 734 is particularly preferred. Further cationic polymers are for example the copolymer of polyvinyl pyrrolidone and imidazolimine methochloride, which is sold by BASF, Germany under the trade name Luviquat® HM 550, the terpolymer of dimethyl diallyl ammonium chloride, sodium acrylate and acrylamide, which is sold by Calgon/USA under the trade name Merquat® Plus 3300, and the vinyl pyrrolidone/methacrylamidopropyl trimethylammonium chloride copolymer sold by ISP under the trade name Gafquat® HS 100.

Homopolymers of the general formula (P1),

{CH₂—[CR¹COO—(CH₂)ₘN⁺R²R³R⁴]}ₙX⁻ (P1)

in which R¹=—H or —CH₃,
R², R³ and R⁴ are selected independently of one another from C1-4 alkyl, alkenyl or hydroxyalkyl groups,
m=1, 2, 3 or 4,
n is a natural number and
X⁻ is a physiologically tolerable organic or inorganic anion, and copolymers consisting substantially of the monomer units included in formula (Monomer-3) along with non-ionogenic monomer units, are particularly preferred cationic polymers. In the context of these polymers those for which at least one of the following conditions applies are preferred according to the invention:
R1 denotes a methyl group
R2, R3 and R4 denote methyl groups
m has the value 2.

Suitable physiologically tolerable counterions X− are for example halide ions, sulfate ions, phosphate ions, methosulfate ions as well as organic ions such as lactate, citrate, tartrate and acetate ions. Halide ions, in particular chloride, are preferred.

A particularly suitable homopolymer is the poly(methacryloyloxyethyl trimethylammonium chloride) with the INCI name Polyquaternium-37. Such products are commercially available for example under the names Rheocare® CTH (Cosmetic Rheologies) and Synthalen® CR (3V Sigma). The homopolymer is preferably used in the form of a non-aqueous polymer dispersion which should have a polymer content of not less than 30 wt. %. Such polymer dispersions are commercially available under the names Salcare® SC 95 and Salcare® SC 96.

Suitable cationic polymers that are derived from natural polymers are cationic derivatives of polysaccharides, for example cationic derivatives of cellulose, starch or guar. Also suitable are chitosan and chitosan derivatives. Cationic polysaccharides have the general formula (P-3) G-O—B—N+RₐR_{b}R_{c} X⁻

G is an anhydroglucose residue, for example starch or cellulose anhydroglucose;
B is a divalent group of compounds, for example alkylene, oxyalkylene, polyoxyalkylene or hydroxyalkylene;
Rₐ, R_{b} and R_{c} are independently of one another alkyl, aryl, alkylaryl, arylalkyl, alkoxyalkyl or alkoxyaryl, each having up to 18 C atoms, the total number of C atoms in
Rₐ, R_{b} and R_{c} preferably being a maximum of 20;
X⁻ is a conventional counterion and is preferably chloride.

A cationic cellulose is sold under the name Polymer JR® 400 by Amerchol and has the INCI name Polyquaternium-10. A further cationic cellulose has the INCI name Polyquaternium-24 and is sold under the trade name Polymer LM-200 by Amerchol. Further commercial products are the compounds Celquat® H 100, and Celquat® L 200. A most highly preferred cellulose is Polyquaternium-67. The cited commercial products are preferred cationic celluloses.

Suitable cationic guar derivatives are sold under the trade name Jaguar and have the INCI name Guar Hydroxypropyltrimonium Chloride. Particularly suitable cationic guar derivatives are also sold furthermore by Hercules under the name N-Hance®. Further cationic guar derivatives are sold by Cognis under the name Cosmedia®. A preferred cationic guar derivative is the commercial product AquaCat® from Hercules. This raw material is a pre-dissolved cationic guar derivative.

Chitosans are a further particularly suitable cationic natural polymer. Along with chitosan itself, quaternized, alkylated and/or hydroxyalkylated derivatives, optionally also in microcrystalline form, are also suitable. They can also be used in the form of aqueous gels with a solids content in the range from 1 to 5 wt. %.

A suitable chitosan is sold for example by Kyowa Oil & Fat, Japan, under the trade name Flonac®. A further chitosan salt is sold under the name Kytamer® PC by Amerchol. Quaternized, alkylated or hydroxyalkylated derivatives, for example hydroxyethyl or hydroxybutyl chitosan, are suitable as chitosan derivatives. Further chitosan derivatives are freely available commercially under the trade names Hydagen® CMF, Hydagen® HCMF and Chitolam® NB/101.

Cationic polymers that are preferred according to the invention are cationic cellulose derivatives and chitosan and derivatives thereof, in particular the commercial products Polymer® JR 400, Hydagen® HCMF and Kytamer PC, cationic guar derivatives, cationic honey derivatives, in particular the commercial product Honeyquat® 50.

These naturally based cationic polymers are contained in the compositions in an amount from 0.01 to 5 wt. %, preferably 0.01 to 3.0 wt. % and particularly preferably in an amount from 0.05 to 3.0 wt. %.

Further preferred cationic polymers are for example
cationic alkyl polyglycosides,
cationized honey, for example the commercial product Honeyquat® 50,
polymeric dimethyl diallyl ammonium salts and copolymers thereof with esters and amides of acrylic acid and methacrylic acid. The products available commercially under the names Merquat®100 (poly(dimethyl diallyl ammonium chloride)) and Merquat®550 (dimethyl diallyl ammonium chloride acrylamide copolymer) are examples of such cationic polymers,
vinyl pyrrolidone-vinyl imidazolium methochloride copolymers, such as are sold under the names Luviquat® FC 370, FC 905 and HM 552,
quaternized polyvinyl alcohol,
and the polymers known under the names Polyquaternium-2, Polyquaternium-17, Polyquaternium-18 and Polyquaternium-27 with quaternary nitrogen atoms in the polymer main chain,
vinyl pyrrolidone-vinyl caprolactam-acrylate terpolymers, such as are available commercially with acrylic acid esters and acrylic acid amides as the third monomer unit under the name Aquaflex® SF 40, for example.

Cationic polymers that are preferred according to the invention are cationic cellulose derivatives and chitosan and derivatives thereof, in particular the commercial products Polymer® JR 400, SofiCAT® with the INCI name Polyquaternium-67, Hydagen® HCMF and Kytamer® PC, cationic guar derivatives, cationic honey derivatives, in particular the commercial product Honeyquat® 50, cationic alkyl polyglycosides and polymers of the Polyquatemium-37 type.

The cationic polymers also include cationized protein hydrolysates, wherein the underlying protein hydrolysate can derive from animal sources, for example from collagen, milk or keratin, from plant sources, for example from wheat, corn, rice, potatoes, soy or almonds, from marine life forms, for example from fish collagen or algae, or from protein hydrolysates obtained by biotechnology. Cationic protein hydrolysates are preferred whose underlying protein component has a molecular weight of 100 to up to 25,000 daltons, preferably 250 to 5000 daltons. Cationic protein hydrolysates are also understood to include quaternized amino acids and mixtures thereof. The cationic protein hydrolysates can furthermore also be further derivatized. Typical examples of the cationic protein hydrolysates and derivatives according to the invention are the products that are listed under the INCI names in the “International Cosmetic Ingredient Dictionary and Handbook”, (seventh edition 1997, The Cosmetic, Toiletry, and Fragrance Association, 1101 17th Street, N.W., Suite 300, Washington, D.C. 20036-4702) and that are available commercially. The plant-based cationic protein hydrolysates and derivatives are most particularly preferred.

The cationic polymers are contained in the compositions according to the invention preferably in amounts from 0.01 to 10 wt. %, relative to the total agent. Amounts from 0.05 to 5 wt. % are particularly preferred.

Like the cationic polymers, amphoteric polymers are likewise most particularly preferred polymers. Amphoteric and/or cationic polymers that are preferred according to the invention are polymers in which a cationic group is derived from at least one of the following monomers:
monomers having quaternary ammonium groups of the general formula (Mono1),

R¹—CH═CR²—CO—Z—(CₙH₂ₙ)—N⁽⁺⁾R³R⁴R⁵A⁽⁻⁾ (Mono1)
in which R¹ and R² independently of one another denote hydrogen or a methyl group and R³, R⁴ and R⁵ independently of one another denote alkyl groups having 1 to 4 carbon atoms, Z is an NH group or an oxygen atom, n is a whole number from 2 to 5 and A⁽⁻⁾ is the anion of an organic or inorganic acid,
monomers having quaternary ammonium groups of the general formula (Mono2),

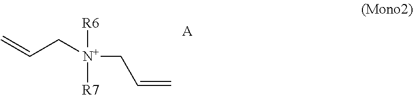

in which R⁶ and R⁷ independently of one another denote a (C₁ to C₄) alkyl group, in particular a methyl group, and

A⁻ is the anion of an organic or inorganic acid.

If a cationic group of the amphoteric or cationic polymers derives from the monomer of formula (Mono1), residues R³, R⁴ and R⁵ in formula (Mono1) preferably denote methyl groups, Z is preferably an NH group and A⁽⁻⁾ preferably denotes a halide, methoxysulfate or ethoxysulfate ion. In this case it is particularly preferable to use acrylamidopropyl trimethylammonium chloride as the monomer (Mono1).

In formula (Mono2) A⁻ preferably denotes a halide ion, in particular chloride or bromide. Preferred amphoteric polymers according to the invention are polymers whose anionic group derives from at least one monomer of formula (Mono3)
monomeric carboxylic acids of general formula (Mono3) or salts thereof with an organic or inorganic acid,

R⁸—CH═CR⁹—COOH (Mono3)
in which R⁸ and R⁹ are independently of one another hydrogen or methyl groups.

Acrylic acid is used as the monomer (Mono3) for the amphoteric polymers preferred according to the invention.

Particularly preferred amphoteric polymers are copolymers of at least one monomer (Mono1) or (Mono2) with the monomer (Mono3), in particular copolymers of monomers (Mono2) and (Mono3). Amphoteric polymers that are most particularly preferably used according to the invention are copolymers of diallyl dimethyl ammonium chloride and acrylic acid. These copolymers are sold under the INCI name Polyquaternium-22, inter alia under the trade name Merquat® 280 (Nalco).

In addition to a monomer (Mono1) or (Mono2) and a monomer (Mono3), the amphoteric polymers according to the invention can moreover additionally contain a monomer (Mono4)
monomeric carboxylic acid amides of the general formula (Mono4),

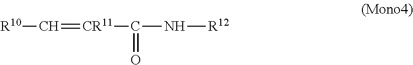

in which R¹⁰ and R¹¹ are independently of one another hydrogen or methyl groups and R¹² denotes a hydrogen atom or a (C₁ to C₈) alkyl group.

Amphoteric polymers based on a comonomer (Mono4) that are most particularly preferably used according to the invention are terpolymers of diallyl dimethyl ammonium chloride, acrylamide and acrylic acid. These copolymers are sold under the INCI name Polyquaternium-39, inter alia under the trade name Merquat® Plus 3330 (Nalco).

Amphoteric polymers which are particularly preferably used are polymers which are composed substantially of

(i) monomers having quaternary ammonium groups of the general formula (Mono1),

R¹—CH═CR²—CO—Z—(CₙH₂ₙ)—N⁽⁺⁾)R²R³R⁴A⁽⁻⁾ (Mono1)
in which R¹ and R² independently of one another denote hydrogen or a methyl group and R³, R⁴ and R⁵ independently of one another denote alkyl groups having 1 to 4 carbon atoms, Z is an NH group or an oxygen atom, n is a whole number from 2 to 5 and A⁽⁻⁾ is the anion of an organic or inorganic acid, and
   (ii) monomeric carboxylic acids of the general formula (Mono3),

R⁸—CH═CR⁹—COOH (Mono3)
in which R8 and R9 are independently of one another hydrogen or methyl groups.

Polymers in which monomers of type (i) are used, in which R³, R⁴ and R⁵ are methyl groups, Z is an NH group and A⁽⁻⁾ is a halide, methoxysulfate or ethoxysulfate ion, are most particularly preferred; acrylamidopropyl trimethylammonium chloride is a particularly preferred monomer (i). Acrylic acid is preferably used as the monomer (ii) for the cited polymers.

The amphoteric polymers can generally be used according to the invention both directly and in the form of the salt, which is obtained by neutralization of the polymers, with an alkali hydroxide for example.

The amphoteric polymers are contained in the agents according to the invention preferably in amounts from 0.01 to 10 wt. %, relative to the total agent. Amounts from 0.01 to 5 wt. % are particularly preferred.

The preferred cationic polymeric compounds according to the invention, as previously described, are selected in particular from cationic celluloses, cationic guar derivatives, cationic starches and the Salcare and Merquat types. The particularly preferred cationic polymeric compounds are Polyquaternium-37, Polyquaternium-80, Polyquaternium-22, Polyquatemium-10, Polyquatemium-11 and Polyquatemium-16.

In the most highly preferred embodiments of the present invention at least one of the aforementioned cationic polymeric compounds is used in each case as an ingredient. The embodiments are most highly preferred when Polyquaternium-37 or Polyquaternium-10 or both together are used as the cationic polymer.

Cosmetic oils can furthermore additionally be used with the active ingredient combination (A) according to the invention. These oil bodies preferably have a melting point below 50° C., particularly preferably below 45° C., most particularly preferably below 40° C., most highly preferably below 35° C. and most preferably the cosmetic oils are free-flowing at a temperature below 30° C. These oils are defined and described in more detail below.

The natural and synthetic cosmetic oils include, for example:
vegetable oils. Examples of such oils are sunflower oil, olive oil, soybean oil, rapeseed oil, almond oil, jojoba oil, orange oil, wheat germ oil, peach kernel oil and the liquid components of coconut butter. Other triglyceride oils are also suitable, however, such as the liquid components of beef fat and synthetic triglyceride oils
liquid paraffin oils, isoparaffin oils and synthetic hydrocarbons and also di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. The compounds 1,3-di-(2-ethylhexyl)cyclohexane and di-n-octyl ether, which are available as commercial products (Cetiol® S and Cetiol® OE respectively), can be preferred.

Suitable natural oils are for example amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, hazelnut oil, elderberry seed oil, blackcurrant seed oil, jojoba oil, cocoa butter, linseed oil, macadamia nut oil, corn oil, almond oil, manila oil, evening primrose oil, olive oil, palm oil, rapeseed oil, rice oil, sea buckthorn fruit oil, sea buckthorn seed oil, sesame oil, shea butter, soybean oil, sunflower oil, grape seed oil, walnut oil or wild rose oil.

The amount of natural and synthetic cosmetic oil bodies used in the agents used according to the invention is conventionally 0.1 to 30 wt. %, relative to the total agent, preferably 0.1 to 20 wt. % and in particular 0.1 to 15 wt. %.

In many cases the agents contain at least one surface-active substance, with both anionic and zwitterionic, ampholytic, non-ionic and cationic surface-active substances being suitable in principle. The choice of surface-active substances is governed by the type of agent. In the case of a shampoo, at least one surfactant from the group of anionic, zwitterionic or non-ionic surface-active substances is chosen in particular. It is preferable here for at least one anionic and at least one zwitterionic surface-active substance to be chosen. These surface-active substances are particularly preferably chosen from the group of particularly gentle surface-active substances.

If the agent is a hair-conditioning composition, then cationic and/or non-ionic surface-active substances are preferably chosen as surface-active substances.

Surface-active substances substantially encompass two groups, surfactants and emulsifiers, which are treated together here.

All anionic surface-active substances that are suitable for use on the human body are suitable as anionic surfactants and emulsifiers (Tanion) in preparations according to the invention. Typical examples of particularly preferred anionic surfactants are:
linear and branched fatty acids having 8 to 30 C atoms (soaps),
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group,
alkyl sulfates and/or alkyl ether sulfates,
esters of tartaric acid and citric acid with alcohols that are addition products of around 2 to 15 molecules of ethylene oxide and/or propylene oxide with fatty alcohols having 8 to 22 C atoms,
alkyl and/or alkenyl ether phosphates of the formula

R¹(OCH₂CH₂)ₙ—O—(PO—OX)—OR²,
in which R¹ preferably denotes an aliphatic hydrocarbon residue having 8 to 30 carbon atoms, R² denotes hydrogen, a (CH₂CH₂O)ₙR² residue or X, n denotes numbers from 1 to 10 and X denotes hydrogen, an alkali or alkaline-earth metal or NR³R⁴R⁵R⁶, with R³ to R⁶ independently of one another denoting hydrogen or a C₁ to C₄ hydrocarbon residue,
monoglyceride sulfates and monoglyceride ether sulfates of the formula

R⁸OC—(OCH₂CH₂)ₓ—OCH₂—[CHO(CH₂CH₂O)_{y}H]—CH₂O(CH₂CH₂O)_{z}—SO₃X,
in which R⁸CO denotes a linear or branched acyl residue having 6 to 22 carbon atoms, x, y and z in total denote 0 or numbers from 1 to 30, preferably 2 to 10, and X denotes an alkali or alkaline-earth metal. Typical examples of suitable monoglyceride (ether) sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates are preferably used in which R⁸CO denotes a linear acyl residue having 8 to 18 carbon atoms,
amide ether carboxylic acids, R¹—CO—NR²—CH₂CH₂—O—(CH₂CH₂O)ₙCH₂COOM, with R1 as a straight-chain or branched alkyl or alkenyl residue having a number of carbon atoms in the chain from 2 to 30, n denotes a whole number from 1 to 20 and R² denotes hydrogen, a methyl, ethyl, propyl, isopropyl, n-butyl, t-butyl or isobutyl residue and M denotes hydrogen or a metal such as alkali metal, in particular sodium, potassium, lithium, alkaline-earth metal, in particular magnesium, calcium, zinc, or an ammonium ion, such as ⁺NR³R⁴R⁵R⁶, with R³ to R⁶ independently of one another denoting hydrogen or a C1 to C4 hydrocarbon residue. Such products are available for example from Chem-Y under the product name Akypo®,
acyl glutamates of the formula XOOC—CH₂CH₂CH(C(NH)OR)—COOX, in which RCO denotes a linear or branched acyl residue having 6 to 22 carbon atoms and 0 and/or 1, 2 or 3 double bonds and X denotes hydrogen, an alkali and/or alkaline-earth metal, ammonium, alkyl ammonium, alkanol ammonium or glucammonium,
condensation products of a water-soluble salt of a water-soluble protein hydrolysate with a C8-C30 fatty acid. Such products have long been available commercially under the trademark Lamepon, Maypon, Gluadin®, Hostapon® KCG or Amisoft®,
alkyl and/or alkenyl oligoglycoside carboxylates, sulfates, phosphates and/or isethionates, and
acyl lactylates.

Particularly suitable zwitterionic surfactants and emulsifiers are the betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name Cocamidopropyl Betaine.

Ampholytic surfactants and emulsifiers (Tampho) are understood to be surface-active compounds that are capable of forming internal salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Typical examples of amphoteric or zwitterionic surfactants are alkyl betaines, alkyl amido betaines, aminopropionates, aminoglycinates, imidazolinium betaines and sulfobetaines.

Particularly preferred ampholytic surfactants and emulsifiers are N-cocoalkyl aminopropionate, cocoacylaminoethyl aminopropionate and C₁₂-C₁₈ acyl sarcosine.

Non-ionic surfactants (Tnio) and non-ionic emulsifiers are for example
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol® types (Cognis),
alkoxylated triglycerides,
amine oxides,
sorbitan fatty acid esters and addition products of ethylene oxide with sorbitan fatty acid esters such as for example polysorbates,
sugar fatty acid esters and addition products of ethylene oxide with sugar fatty acid esters,
polyglycerols and polyglycerol derivatives such as for example polyglycerol poly-12-hydroxystearate (commercial product Dehymuls® PGPH),
sugar surfactants of the alkyl and alkenyl oligoglycoside type, and
sugar surfactants of the fatty acid-N-alkyl polyhydroxyalkylamide type.

In addition to the cationic compounds according to claim 1 a), all conventional cationic surfactants known to the person skilled in the art can be used according to the invention as cationic surfactants. These are in particular
cationic surfactants according to formula (Tkat-2),

RCO—X—N⁺R¹R²R³A⁻ (Tkat-2)
R here denotes a substituted or unsubstituted, branched or straight-chain alkyl or alkenyl residue having 11 to 35 carbon atoms in the chain, X denotes —O— or —NR⁵R¹ denotes an alkylene group having 2 to 6 C atoms, which can be non-substituted or substituted, wherein if it is substituted, substitution with an —OH or —NH group is preferred,
R², R³ independently of one another denote an alkyl or hydroxyalkyl group having 1 to 6 C atoms in the chain, wherein the chain can be straight or branched,
R⁵ denotes hydrogen or a C1 to C6 straight-chain or branched alkyl or alkenyl residue, which can also be substituted with a hydroxyl group. Within this structural class, compounds of one of the following structures are preferably used:

CH₃(CH₂)₂₀CONH(CH₂)₃—N⁺(CH₃)₂—CH₂CH₃A⁽⁻⁾ (Tkat-3)

CH₃(CH₂)₂₀CONH(CH₂)₃—N⁺(CH₃)₂—CH₂(CHOH)CH₂OHA⁽⁻⁾ (Tkat-4)

CH₃(CH₂)₂₀COOCH₂CHOHCH₂—N⁺(CH₃)₃A⁽⁻⁾ (Tkat-5)

CH₃(CH₂)₂₀CONH(CH₂)₃—N⁺(CH₃)₂—CH₂CH₂OHA⁽⁻⁾ (Tkat-6)
Examples of such commercial products are Schercoquat BAS, Lexquat AMG-BEO, Akypoquat 131 or Incroquat Behenyl HE.
Esterquats according to formula (Tkat1-2) can be used.

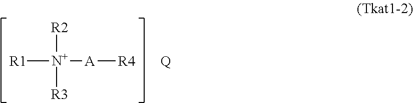

Here residues R1, R2 and R3 are independent of one another and can be identical or different. Residues R1, R2 and R3 denote:
   a branched or unbranched alkyl residue having 1 to 4 carbon atoms, which can contain at least one hydroxyl group, or
   a saturated or unsaturated, branched or unbranched or a cyclic saturated or unsaturated alkyl residue having 6 to 30 carbon atoms, which can contain at least one hydroxyl group, or
   an aryl or alkaryl residue, for example phenyl or benzyl,
   the residue (-A-R4), with the proviso that at most 2 of residues R1, R2 or R3 can denote this residue.
   The residue -(A-R4) is included at least 1 to 3 times.
   Here A denotes:
   1) —(CH2)n- where n=1 to 20, preferably n=1 to 10 and particularly preferably n=1-5, or
   2) —(CH2-CHR5-O)n- where n=1 to 200, preferably 1 to 100, particularly preferably 1 to 50, and particularly preferably 1 to 20 with R5 denoting hydrogen, methyl or ethyl, and
   R4 denotes:
   1) R6-O—CO—, in which R6 is a saturated or unsaturated, branched or unbranched or a cyclic saturated or unsaturated alkyl residue having 6 to 30 carbon atoms, which can contain at least one hydroxyl group and which can optionally also be ethoxylated with 1 to 100 ethylene oxide units and/or 1 to 100 propylene oxide units, or
   2) R7-CO—, in which R7 is a saturated or unsaturated, branched or unbranched or a cyclic saturated or unsaturated alkyl residue having 6 to 30 carbon atoms, which can contain at least one hydroxyl group and which can optionally also be ethoxylated with 1 to 100 ethylene oxide units and/or 1 to 100 propylene oxide units, and
   Q denotes a physiologically tolerable organic or inorganic anion.
   Such products are sold under the trademarks Rewoquat®, Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of such esterquats.
   Other compounds of formula (Tkat1-2) that are particularly preferred according to the invention are included in formula (Tkat1-2.1), cationic betaine esters.

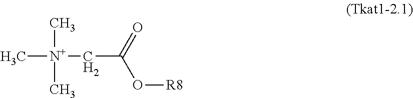

R8 corresponds in its meaning to R7.
monoalkyl trimethylammonium salts with a chain length of the alkyl residue of 16 to 24 carbon atoms, corresponding to formula (Tkat1-1),

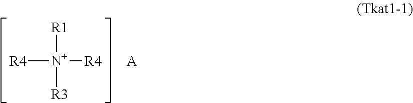

in which R1, R2 and R3 each denote a methyl group and R4 denotes a saturated, branched or unbranched alkyl residue having a chain length of 16 to 24 carbon atoms. Examples of compounds of formula (Tkat1-1) are cetyl trimethylammonium chloride, cetyl trimethylammonium bromide, cetyl trimethylammonium methosulfate, stearyl trimethylammonium chloride, behenyl trimethylammonium chloride, behenyl trimethylammonium bromide and behenyl trimethylammonium methosulfate. In most highly preferred compositions according to the invention the latter is used together with a cationic compound according to claim 1 a) in amounts as described below.
amines and/or cationized amines, in particular an amidoamine and/or a cationized amidoamine of the following structural formulae:

R¹—NH—(CH₂)ₙ—NR²R³ (Tkat7)

and/or

R¹—NH—(CH₂)ₙ—NR²R³R⁴ (Tkat8)
in which R1 denotes an acyl or alkyl residue having 6 to 30 C atoms, which can be branched or unbranched, saturated or unsaturated, and wherein the acyl residue and/or alkyl residue can contain at least one OH group, and
R2, R3 and R4 independently of one another denote hydrogen or an alkyl residue having 1 to 4 C atoms, which can be identical or different, saturated or unsaturated, and
X⁻ denotes an anion and
n denotes a whole number between 1 and 10.

A composition in which the amine and/or the quaternized amine according to the general formulae (Tkat7) and/or (Tkat8) is an amidoamine and/or a quaternized amidoamine is preferred, in which R1 denotes a branched or unbranched, saturated or unsaturated acyl residue having 6 to 30 C atoms, which can contain at least one OH group. A fatty acid residue from oils and waxes, in particular from natural oils and waxes, is preferred here. Suitable examples include lanolin, beeswax or candelilla wax. Also preferred are amidoamines and/or quaternized amidoamines in which R2, R3 and/or R4 in formulae (Tkat7) and/or (Tkat8) denote a residue according to the general formula CH₂CH₂OR5, in which R5 can have the meaning of alkyl residues having 1 to 4 carbon atoms, hydroxyethyl or hydrogen. The preferred value of n in the general formulae (Tkat7) and/or (Tkat8) is a whole number between 2 and 5. Also preferred are amidoamines and/or quaternized amidoamines of the general formulae (Tkat7) and/or (Tkat8) in which the anion X⁻ is a halide ion or a compound of the general formula RSO₃⁻, in which R has the meaning of saturated or unsaturated alkyl residues having 1 to 4 carbon atoms. The alkyl residue having 1 to 4 carbon atoms of R2, R3 and R4 and/or the alkyl residue having 1 to 4 carbon atoms of RSO₃⁻ in the general formula (Tkat7) and/or (Tkat8) can contain at least one hydroxyl group. The alkyl amidoamines can be present as is and can also be converted into a quaternary compound in the composition by protonation in a correspondingly acid solution. Cationic alkyl amidoamines are preferred according to the invention.

Suitable amidoamines for use according to the invention, which can optionally be quaternized, include for example: Witcamine® 100 (Witco, INCI name: Cocamidopropyl Dimethylamine), Incromine® BB (Croda, INCI name: Behenamidopropyl Dimethylamine), Mackine® 401 (McIntyre, INCI name: Isostearylamidopropyl Dimethylamine) and other Mackine types, Adogen® S18V (Witco, INCI name: Stearylamidopropyl Dimethylamine), and as permanently cationic aminoamines: Rewoquat® RTM 50 (Witco Surfactants GmbH, INCI name: Ricinoleamidopropyltrimonium Methosulfate), Empigen® CSC (Albright & Wilson, INCI name: Cocamidopropyltrimonium Chloride), Swanol® Lanoquat DES-50 (Nikko, INCI name: Quaternium-33), Rewoquat® UTM 50 (Witco Surfactants GmbH, Undecyleneamidopropyltrimonium Methosulfate).

The anion of all cationic compounds described above is selected from the physiologically tolerable anions. The halide ions, fluoride, chloride, bromide, sulfate of the general formula RSO₃⁻, in which R has the meaning of saturated or unsaturated alkyl residues having 1 to 4 carbon atoms, or anionic residues of organic acids such as maleate, fumarate, oxalate, tartrate, citrate, lactate or acetate, are cited by way of example.

Cationic imidazolines, esterquats, cationic surfactants according to formula (Tkat-2) and amines and/or cationized amines, in particular amidoamines and/or cationized amidoamines are preferably used.

The aforementioned cationic surfactants can be used individually or in any combinations with one another, wherein they are included in amounts of between 0.01 and 20 wt. %, preferably in amounts from 0.01 to 10 wt. % and most particularly preferably in amounts from 0.1 to 7.5 wt. %. The very best results are obtained with amounts from 0.1 to 5 wt. %, relative in each case to the overall composition of the individual agent.

The surfactants (T) are used in a total amount of surfactants in amounts from 0.05 to 45 wt. %, preferably 0.1 to 30 wt. % and most particularly preferably from 0.5 to 25 wt. %, relative to the total agent used according to the invention.

Most particularly preferred compositions according to the invention according to claim 1 contain furthermore at least one carbohydrate, a sugar alcohol or sugar. Monosaccharides, disaccharides, trisaccharides and oligosaccharides, amino deoxy sugars, deoxy sugars, thio-sugars are advantageous in particular. Of these, monosaccharides having 3 to 8 C atoms, such as for example trioses, tetroses, pentoses, hexoses, heptoses and octoses, are most particularly preferred. Oligosaccharides having up to 50 monomer units are moreover most highly preferred. Sorbitol, inositol, mannitol, tetritols, pentitols, hexitols, threitol, erythritol, adonitol, arabitol, xylitol, dulcitol, erythrose, threose, arabinose, ribose, xylose, lyxose, glucose, galactose, mannose, allose, altrose, gulose, idose, talose, fructose, sorbose, psicose, tegatose, deoxyribose, glucosamine, galactosamine, rhamnose, digitoxose, thioglucose, sucrose, trehalose, lactose, maltose, cellobiose, melibiose, gestiobiose, rutinose, raffinose and cellotriose are mentioned by way of example. Preferred carbohydrates are sorbitol, inositol, mannitol, threitol, erythreitol, erythrose, threose, arabinose, ribose, xylose, glucose, galactose, mannose, allose, fructose, sorbose, deoxyribose, glucosamine, galactosamine, sucrose, trehalose, maltose and cellobiose. Glucose, galactose, mannose, fructose, deoxyribose, glucosamine, sucrose, maltose and cellobiose are particularly preferably used. The use of glucose, galactose, lactose, mannose, fructose, sucrose, maltose or cellobiose is however most particularly preferred.

Irrespective of the type of carbohydrate used, compositions according to the invention are preferred which, relative to the weight of the composition, contain 0.01 to 5 wt. %, preferably 0.05 to 4 wt. %, particularly preferably 0.05 to 3.5 wt. % and in particular 0.1 to 2.5 wt. % of carbohydrate.

Preferred compositions according to the invention according to claim 1 furthermore contain vitamins, provitamins or vitamin precursors in the compositions.

Vitamins, provitamins and vitamin precursors are particularly preferred that are assigned to groups A, B, C, E, F and H.

The group of substances classed as vitamin A includes retinol (vitamin A₁) and 3,4-didehydroretinol (vitamin A₂). β-Carotene is the retinol provitamin. Suitable vitamin A components according to the invention are for example vitamin A acid and esters thereof, vitamin A aldehyde and vitamin A alcohol and esters thereof such as the palmitate and acetate. The agents according to the invention contain the vitamin A component preferably in amounts from 0.05 to 1 wt. %, relative to the overall preparation.

The vitamin B group or vitamin B complex includes inter alia

Vitamin B₁ (thiamine)

Vitamin B₂ (riboflavin)

Vitamin B₃. The compounds nicotinic acid and nicotinic acid amide (niacinamide) are often included under this term. Preferred according to the invention is nicotinic acid amide, which is preferably contained in the agents used according to the invention in amounts from 0.05 to 1 wt. %, relative to the total agent.

Vitamin B₅ (pantothenic acid, panthenol and pantolactone). Within the context of this group panthenol and/or pantolactone is preferably used. Derivatives of panthenol which can be used according to the invention are in particular the esters and ethers of panthenol as well as cationically derivatized panthenols. Individual representatives are for example panthenol triacetate, panthenol monoethyl ether and the monoacetate thereof, and cationic panthenol derivatives. The cited compounds of the vitamin B₅ type are preferably contained in the agents according to the invention in amounts from 0.05 to 10 wt. %, relative to the total agent. Amounts from 0.1 to 5 wt. % are particularly preferred.

Vitamin B₆ (pyridoxine as well as pyridoxamine and pyridoxal).

Of the vitamins of the B series, vitamins of the B3 series and B5 series are particularly preferred.

Vitamin C (ascorbic acid). Vitamin C is used in the agents according to the invention preferably in amounts from 0.1 to 3 wt. %, relative to the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular esters such as acetate, nicotinate, phosphate and succinate, are preferably contained in the agents according to the invention in amounts from 0.05 to 1 wt. %, relative to the total agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably contained in the agents according to the invention in amounts from 0.0001 to 1.0 wt. %, in particular in amounts from 0.001 to 0.01 wt. %.

The compositions according to the invention preferably contain vitamins, provitamins and vitamin precursors from groups A, B, E and H. Vitamin B and biotin are most particularly preferred.

The viscosity of the compositions according to the invention can be adjusted using all raw materials known for that purpose. It is however preferable according to the invention for in particular non-ionic, naturally based polymers to be chosen for adjusting the viscosity. These are in particular xanthan gums, for example Keltrol® types, guar gums, gelatins, pectins, starches, for example from potatoes, rice or corn. Starch in particular is used in a pre-gelatinized form. Such products are commercially available from Avebe, Hercules, National Starch and others. The non-ionic, naturally based polymers are contained in the compositions according to the invention in an amount from 0.01 to 10 wt. %, preferably 0.01 to 7.5 wt. %, particularly preferably 0.05 to 5.0 wt. %, relative in each case to the weight of the overall composition.

Further ingredients of the compositions according to claim 1 can be all further ingredients conventionally used in cosmetic agents. The person skilled in the art will choose them selectively, according to the purpose of the agent. Some most particularly preferred further ingredients are described below by way of example.

The first group of further ingredients are plant extracts. Preferred above all according to the invention are the extracts from green tea, oak bark, stinging nettle, witch hazel, hops, henna, chamomile, burdock, horsetail, whitethom, lime blossom, almond, aloe vera, pine, horse chestnut, sandalwood, juniper, coconut, mango, apricot, acai berry, cranberry, lemon, wheat, kiwi, melon, orange, grapefruit, sage, rosemary, birch, mallow, valerian, lady's smock, wild thyme, yarrow, thyme, melissa, restharrow, coltsfoot, marshmallow, meristem, ginseng, coffee, cocoa, moring a, ginger root and ayurvedic plant extracts such as for example Aegle marmelos (bilva), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamonum zeylanicum and Nelumbo nucifera (kamala), grasses such as wheat, barley, rye, oats, spelt, corn, the various types of millet (proso millet, finger millet, foxtail millet as examples), sugar cane, ryegrass, meadow foxtail, false oat-grass, bentgrass, meadow fescue, moor grass, bamboo, cottongrass, pennisetums, Andropogonodeae (Imperata cylindrica, also known as blood grass or cogon grass), buffalo grass, cord grass, dog's tooth grass, lovegrass, Cymbopogon (citronella grass), Oryzeae (rice), Zizania (wild rice), marram grass, blue oatgrass, soft-grass, quaking grasses, speargrass, couch grass and Echinacea, in particular Echinacea angustifolia DC, Echinacea paradoxa (Norton), Echinacea simulata, E. atrorubens, E. tennesiensis, Echinacea strigosa (McGregor), Echinacea laevigata, Echinacea purpurea (L.) Moench and Echinacea pallida (Nutt), all types of vine and pericarp of Litchi chinensis.

The plant extracts can be used according to the invention in both pure and diluted form. Relative to the content of the individual plant extract, plant extracts are used in amounts such that active ingredient contents of the individual extract are contained in the compositions according to the invention in an amount from 0.001 to 7.5 wt. %, preferably 0.01 to 5.0 wt. % and particularly preferably from 0.01 to 3.0 wt. %.

Protein hydrolysates can of course additionally be used according to the invention. To this end protein hydrolysates of both plant and animal or marine or synthetic origin can be used.

Animal protein hydrolysates are for example elastin, collagen, keratin, silk and milk protein hydrolysates, which can also be present in the form of salts. Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Also preferred according to the invention are plant protein hydrolysates, for example soy, almond, pea, moring a, potato and wheat protein hydrolysates. Such products are available for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Sérobiologiques.

Further protein hydrolysates that are preferred according to the invention are of marine origin. They include for example collagen hydrolysates of fish or algae and protein hydrolysates of mussels or pearl hydrolysates. Examples of pearl extracts according to the invention are the commercial products Pearl Protein Extract BG® or Crodarom® Pearl.

The protein hydrolysates (P) are contained in the compositions in concentrations from 0.001 wt. % to 20 wt. %, preferably from 0.05 wt. % to 15 wt. % and most particularly preferably in amounts from 0.05 wt. % to 5 wt. %.

A particularly preferred group of ingredients in the cosmetic compositions according to the invention is constituted by the betaines listed below: carnitine, carnitine tartrate, carnitine magnesium citrate, acetyl carnitine, betalains, 1,1-dimethyl proline, choline, choline chloride, choline bitartrate, choline dihydrogen citrate and the compound N,N,N-trimethylglycine, which is classed in the literature as betaine.

Carnitine, histidine, choline and betaine are preferably used. In a particularly preferred embodiment of the invention L-carnitine tartrate is used as the active ingredient.

Agents which, relative to their weight, contain 0.00001 to 10.0 wt. %, preferably 0.0001 to 5.0 wt. % and in particular 0.001 to 3 wt. % of the cited betaines are particularly preferred according to the invention.

A particularly substantial ingredient is taurine and/or a taurine derivative. Taurine is understood to be exclusively 2-aminoethane sulfonic acid, and a derivative to be the explicitly cited taurine derivatives. The taurine derivatives are understood to be N-monomethyl taurine, N,N-dimethyltaurine, taurine lysylate, taurine tartrate, taurine ornithate, lysyl taurine and omithyl taurine. Further taurine derivatives within the meaning of the present invention are taurocholic acid and hypotaurine.

Agents according to the invention which, relative to their weight, contain 0.0001 to 10.0 wt. %, preferably 0.0005 to 5.0 wt. %, particularly preferably 0.001 to 2.0 wt. % and in particular 0.001 to 1.0 wt. % of taurine and/or a taurine derivative are particularly preferred.

In a further embodiment that is preferred according to the invention the compositions according to the invention contain bioquinones. In the agents according to the invention suitable bioquinones are understood to be one or more ubiquinones and/or plastoquinones. The ubiquinones that are preferred according to the invention have the following formula:

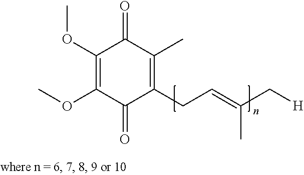

Coenzyme Q-10 is most preferred here.

Preferred compositions according to the invention contain purine and/or purine derivatives in relatively narrow quantity ranges. Cosmetic agents that are preferred according to the invention have the characterizing feature that they contain, relative to their weight, 0.001 to 2.5 wt. %, preferably 0.0025 to 1 wt. %, particularly preferably 0.005 to 0.5 wt. % and in particular 0.01 to 0.1 wt. %.

Preferred compositions according to the invention contain purine and/or purine derivatives in relatively narrow quantity ranges. Cosmetic agents that are preferred according to the invention have the characterizing feature that they contain, relative to their weight, 0.001 to 2.5 wt. %, preferably 0.0025 to 1 wt. %, particularly preferably 0.005 to 0.5 wt. % and in particular 0.01 to 0.1 wt. % of purine(s) and/or purine derivative(s).

Some representatives of purine, purines and purine derivatives are particularly preferred according to the invention. Cosmetic agents that are preferred according to the invention have the characterizing feature that they contain at least one of the following purines: purine, adenine, guanine, uric acid, hypoxanthine, 6-purinethiol, 6-thioguanine, xanthine, caffeine, theobromine or theophylline.

Depending on the desired application of the cosmetic agents, the nature and amount of the purine derivative can vary. In hair cosmetics formulations, caffeine, which can be used in shampoos, conditioners, hair tonics and/or lotions for example, preferably in amounts from 0.005 to 0.25 wt. %, more preferably from 0.01 to 0.1 wt. % and in particular from 0.01 to 0.05 wt. % (relative in each case to the composition), has proved effective in particular.

Linear and/or branched, saturated and/or unsaturated fatty acids having 6 to 30 carbon atoms can be used as fatty acids (Fatac). Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all other fatty acids sold under the Edenor® trade names (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. The fatty acid cuts obtainable from coconut oil or palm oil are conventionally particularly preferred; as a rule the use of stearic acid is preferred in particular.

The amount used here is 0.1 to 15 wt. %, relative to the total agent. The amount is preferably 0.5 to 10 wt. %, wherein amounts of 1 to 5 wt. % can be most particularly advantageous.

Saturated, mono- or polyunsaturated, branched or unbranched fatty alcohols having C₆ to C₃₀, preferably C₁₀ to C₂₂ and most particularly preferably C₁₂ to C₂₂ carbon atoms can be used as fatty alcohols (Fatal). Suitable for use within the meaning of the invention are for example decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as the Guerbet alcohols thereof, wherein this list is intended to be of an exemplary and non-limiting nature. However, the fatty alcohols derive from preferably natural fatty acids, wherein it can conventionally be assumed that they are obtained from the esters of fatty acids by reduction. Likewise suitable for use according to the invention are fatty alcohol cuts constituting a mixture of different fatty alcohols. Such substances are available commercially for example under the names Stenol®, e.g. Stenol® 1618 or Lanette®, e.g. Lanefte® 0 or Lorol®, e.g. Lorol® C8, Lorol® C14, Lorol© C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are available commercially for example under the names Corona®, White Swan®, Coronet® or Fluilan®. The fatty alcohols are used in amounts from 0.1 to 30 wt. %, relative to the overall preparation, preferably in amounts from 0.1 to 20 wt. %.

Solid paraffins or isoparaffins, carnauba waxes, beeswaxes, candelilla waxes, ozokerites, ceresin, spermaceti wax, sunflower wax, fruit waxes such as for example apple wax or citrus wax, PE or PP microwaxes can be used according to the invention as natural or synthetic waxes (Fatwax). Such waxes are available for example from Kahl & Co., Trittau.

The cosmetic agents can furthermore contain additional active ingredients, auxiliary substances and additives, such as for example
UV light-screening filters having an effect in the range of UV-A, UV-B and UV-C light, both as a water-soluble and oil-soluble filter,
further cationic, amphoteric, anionic or non-ionic polymers on a synthetic basis, in particular vinyl pyrrolidone/vinyl ester copolymers, such as are sold for example as Luviskol® VA 64 and Luviskol® VA 73,
cellulose ethers, such as hydroxypropyl cellulose, hydroxyethyl cellulose and methylhydroxypropyl cellulose, such as are sold for example under the trademarks Culminal® and Benecel® (AQUALON), and Natrosol® types (Hercules),
starch and derivatives thereof, in particular starch ethers, for example Structure® XL (National Starch) or Structure® ZEA, multifunctional, salt-tolerant starches,
shellac,
dye precursors of the coupler and developer type,
substantive dyes,
texturizing agents such as maleic acid and lactic acid,
swelling agents such as urea, allantoin, carbonates or hydantoin,
Dimethyl isosorbide and cyclodextrins,
dyes to color the agent,
anti-dandruff active ingredients such as piroctone olamine, zinc omadine and climbazole,
complexing agents such as EDTA, NTA, β-alanine diacetic acid and phosphonic acids,
opacifiers such as latex, styrene/PVP and styrene/acrylamide copolymers,
pearlescent agents such as ethylene glycol mono- and distearate as well as PEG-3 distearate,
pigments,
stabilizing agents for hydrogen peroxide and other oxidizing agents,
propellants such as propane-butane mixtures, N₂O, dimethyl ether, CO₂ and air,
antioxidants,
perfume oils, scents and fragrances.

With regard to further optional components and to the amounts of these components used, reference is expressly made to the relevant manuals known to the person skilled in the art.

As already mentioned, importance is attached to the good caring effect of the agents according to the invention in particular because they deliver outstanding results even in the presence of oxidizing agents—for example in the context of oxidative hair coloring.

The invention secondly provides a method for hair treatment, in which a cosmetic agent according to claim 1 is applied to the hair and remains on the hair until the next time it is washed. Following application of the composition according to the invention the hair can be dressed and styled by all conventional methods. In particular, the application of heat in the form of a hairdryer, towel, drying hood or curling tongs or curlers is also possible according to the invention. Other compositions supporting styling of the hair, for example a hair spray, can of course also be used.

If desired, however, the composition according to the invention can also be left on the hair for a few seconds to 30 minutes and then rinsed out again.

In the most preferred form according to the invention the composition according to claim 1 is packaged as a spray application. To achieve compositions that are stable in storage over an extended temperature range, for example from −40° C. to +60° C., a viscosity of 2000 to 25,000 mPas, measured at room temperature (23° C.) by the Brookfield method using a number 4 spindle, is preferably established. Adjusting the viscosity is familiar to the person skilled in the art. Conventional polymeric compounds, as previously described, are used to this end for example. Other conventional methods are also possible, however. A viscosity from 5000 mPas to 15,000 mPas is particularly preferred, most highly preferably from 5000 to 10,000 mPas. These viscosities are measured as previously mentioned.

When packaged in the preferred presentation form according to the invention as a non-aerosol spray application, the spray head and spray pump in particular must be specially adapted to the viscosity properties of the composition. Otherwise too much of the composition is discharged, or the spray pattern is too coarse and poorly distributed on the hair, or the spray mist is much too fine, which likewise has a negative influence on the effect of the composition. Advantageously, a special spray pump is individually selected according to the viscosity range of the composition according to the invention. The pump characteristics, such as for example the internal diameter of the nozzle, the pump stroke, the piston volume, the length of the dip tube, the nozzle geometry, etc., must be adapted as necessary. For the viscosity of 5000 to 10,000 mPas that is particularly preferred according to the invention, a Seaquist PZ2 spray pump, for example, with a nozzle having an internal diameter of max. 0.25 mm and a dip tube length of 150 mm and a dip tube internal diameter from 1.0 to 8 mm, preferably from 1 to 5 mm and particularly preferably from 1 to 2.5 mm, has proved to be very suitable.

The present invention therefore thirdly provides a non-aerosol composition for treating keratinic fibers, containing
a) at least 0.01 wt. % of a cationic imidazoline derivative of the 4,5-dihydro-1-(C1 to C4 alkyl)-2-(C12 to C30 alkyl)-1-(2-(C12-C30 alkyl) amidoethyl) imidazolinium salt type, in particular those having a methyl, ethyl, propyl or butyl group as the C1 to C4 alkyl, and having in each case as the C12 to C30 alkyl residue, independently of one another but most highly preferably simultaneously, an alkyl residue of at least 20 C atoms, and particularly preferably 21 C atoms,
b) at least one silicone selected from the dimethicones, dimethiconols and amodimethicones and
c) a cosmetic carrier containing in addition to water an alcohol selected from glycol, glycerol, 1,2-pentanediol, 1,5-pentanediol, 1,2-hexanediol, 1,6-hexanediol, benzyl alcohol, phenoxyethanol or mixtures thereof,
d) wherein the composition has a viscosity of 5000 to 10,000 mPas at 20° C. measured by the Brookfield method using a number 5 spindle, and
e) [is placed] in a container which is large enough for the contents to be discharged with the aid of a 150 ml long dip tube by means of a spray pump fitted with a nozzle with a diameter of max. 0.25 mm.

The examples below are intended to illustrate the subject-matter of the present invention without however limiting it.

### EXAMPLES AND PROOF OF EFFECTIVENESS

Unless otherwise specified, all stated amounts are parts by weight. The following formulations were prepared using known production methods.

### Comparative Examples According to the Prior Art

| | C1 | C2 | C3 |
|---|---|---|---|
| Stearamidopropyl dimethylamine | 1.0 | — | — |
| Polyquaternium-67 | 0.2 | — | — |
| Glycerol | 1.3 | — | — |
| Nicotinic acid amide | 0.15 | — | — |
| D-Panthenol (75%) | 0.2 | 0.25 | 0.25 |
| Silk protein hydrolysate | 0.1 | 0.1 | — |
| Dehyquart ® A-CA | 0.4 | 0.5 | 0.5 |
| Silcare ® Silicone SEA | 0.5 | — | — |
| PEG-40 hydrogenated castor oil | 1.2 | — | — |
| Cetiol ® CG | 0.1 | — | — |
| Ethanol (96%) | 10.0 | — | — |
| Gluadin ® WQ | — | 0.25 | 0.25 |
| Luviquat ® FC 550 | — | 1.0 | 1.0 |
| Pantolactone | — | 0.2 | 0.2 |
| Gluadin ® WLM | — | 0.25 | 0.25 |
| Sericin ® H | — | 0.04 | — |
| Benzophenone-4 | — | 0.05 | 0.05 |
| Amaranth powder (85%) | — | 0.0001 | 0.0001 |
| Dow Corning ® 5225 C Formulation Aid | — | 3.5 | 3.5 |
| Apricot kernel oil | — | 0.2 | 0.2 |
| Cyclomethicone and dimethiconol (15%) | — | 4.5 | 4.5 |
| Dow Corning ® 556 | — | 1.0 | 1.0 |
| Lactic acid (80%) | 1.13 | 0.2 | 0.25 |
| Dye, perfume, preservative, water | to 100 | to 100 | to 100 |
| pH | 3.0-4.0 | 3.1-3.3 | 3.1-3.3 |

### Examples According to the Invention

| | E1 | E2 | E3 | E4 | E5 | E6 |
|---|---|---|---|---|---|---|
| Gluadin ® WQ | — | — | — | — | — | 0.2 |
| D-Panthenol (75%) | 0.2 | 0.2 | 0.2 | 0.2 | 0.2 | 0.2 |
| Silk protein hydrolysate | 0.1 | 0.1 | 0.1 | 0.1 | 0.1 | — |
| Cetearyl alcohol | 1.25 | 1.25 | 1.25 | 1.25 | 2.0 | 1.0 |
| Cutina ® AGS | 2.0 | 2.0 | 2.0 | 2.0 | 2.0 | 2.0 |
| Dehyquart ® F75 | 1.5 | 1.5 | 1.5 | 1.5 | 1.5 | 1.5 |
| Crodazosoft ® DBQ | 1.25 | 1.25 | 1.25 | 1.25 | 1.25 | 2.0 |
| Cetiol ® CG | 0.7 | 0.7 | 0.7 | 0.7 | 0.7 | 0.7 |
| Isopropyl myristate | 2.0 | 2.0 | 2.0 | 2.0 | 2.0 | 2.0 |
| Citric acid | 0.05 | 0.05 | 0.05 | 0.05 | 0.05 | 0.05 |
| Glycerol | 1.0 | 1.0 | 1.0 | 1.0 | 1.0 | 0.7 |
| Phenoxyethanol | 0.4 | 0.4 | 0.4 | 0.4 | 0.4 | 0.4 |
| ProSina ® | — | — | — | — | — | 0.1 |
| Nicotinic acid amide | 0.15 | 0.15 | 0.15 | 0.15 | 0.15 | — |
| Xiameter ® MEM-1664 Emulsion | 2.0 | 2.0 | 4.0 | 2.0 | 3.0 | — |
| Polyquaternium-37 | — | 0.3 | — | 0.2 | — | — |
| Dehyquart ® A-CA | — | — | — | 2.0 | — | — |
| Dye, perfume, preservative, water | to 100 | to 100 | to 100 | to 100 | to 100 | to 100 |
| Viscosity (in mPas, spindle 5, | 5220 | 7700 | 3060 | — | 7340 | 4040 |
| 20° C.) | | | | | | (spindle 4) |
| pH | 3.7 | 3.7 | 3.7 | 3.7 | 3.6 | 2.8-3.4 |

### Proof of Effectiveness

In a side-by-side test, two of the above formulations at a time, one according to the invention and one according to the prior art in each case, were applied to washed and towel-dried hair. The test subjects had highlighted and/or colored hair. Persons skilled in the art who had undergone special sensory training carried out the assessments. They assessed the combability of the wet and dry hair, the feel of the wet and dry hair, the static charge, the volume of the hair, the gloss, the spray behavior of the formulations and the long-term effect, i.e. the result maintained for several subsequent hair washes. The long-term effect is retained for at least one, preferably 3, particularly preferably 5 further hair washes, after several hours.

### Results:

Comparison of C3 with E6:

The formulation E6 according to the invention is judged to be markedly better in all parameters. In particular, the long-term effect, the feel of the wet hair, the static charge and the spray behavior were significantly improved.

Comparison of C3 with E1:

The formulation E1 according to the invention is judged to be significantly better in all parameters. The markedly better results regarding the combability of the wet hair, the static charge, the gloss, the long-term effect and the spray behavior should be emphasized in particular.

Comparison of C3 with E2:

The result is substantially the same as that obtained in the previous comparison.

Comparison of C3 with E3:

Once again the result is substantially the same as that previously obtained.

Comparison of C3 with E4:

Once again the result is the same as that previously obtained.

Comparison of C3 with E5:

As before, the superiority of the composition according to the invention is demonstrated in this case too.

Comparison of C1 with E3:

The composition according to the invention is once again clearly superior to the comparative composition.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for treating human hair, containing
a) at least 0.01 wt. % of a cationic imidazoline derivative of the 4,5-dihydro-1-(C1 to C4 alkyl)-2-(C12 to C30 alkyl)-1-(2-(C12-C30 alkyl) amidoethyl) imidazolinium salt type, in particular those having a methyl, ethyl, propyl or butyl group as the C1 to C4 alkyl, and having in each case as the C12 to C30 alkyl residue, independently of one another but most highly preferably simultaneously, an alkyl residue of at least 20 C atoms, and particularly preferably of at least 21 C atoms,
b) at least one silicone selected from the dimethicones, dimethiconols and amodimethicones and
c) a cosmetic carrier containing in addition to water an alcohol selected from glycol, glycerol, 1,2-pentanediol, 1,5-pentanediol, 1,2-hexanediol, 1,6-hexanediol, benzyl alcohol, phenoxyethanol or mixtures thereof.

2. The cosmetic composition according to claim 1, wherein the cationic imidazoline derivative is Quaternium-91.

3. The cosmetic composition according to claim 1, wherein the silicones selected from the dimethicones, dimethiconols and amodimethicones were prepared by the emulsion polymerization method.

4. The cosmetic composition according to claim 1, wherein the alcohol in the cosmetic carrier is selected from glycerol, benzyl alcohol and phenoxyethanol or mixtures thereof.

5. The agent according to claim 1, wherein furthermore it contains at least one further cationic compound.

6. The agent according to claim 5, wherein the further cationic compound is a cationic polymer.

7. The agent according to claim 1, wherein it additionally contains at least one substance from the group of vitamins, provitamins and vitamin precursors and derivatives thereof, vitamins, provitamins and vitamin precursors assigned to groups B and H being preferred.

8. A method for hair treatment in which a cosmetic agent according to claim 1 is applied to the hair and the hair is further dressed and styled by conventional methods, optionally under exposure to heat, and the agent is only washed out again the next time the hair is washed.
